(19) United States
(12) Patent Application Publication
(10) Pub. No.: US 2024/0416033 A1
(43) Pub. Date: Dec. 19, 2024

(54) SYSTEM AND METHOD OF PAIRING AN INFUSION PUMP WITH A REMOTE CONTROL DEVICE

(71) Applicant: Tandem Diabetes Care, Inc., San Diego, CA (US)

(72) Inventors: Christopher Jung, San Diego, CA (US); Kelly Funk, San Diego, CA (US); Virginia S. Lu, San Diego, CA (US); Jason Farnan, San Diego, CA (US)

(21) Appl. No.: 18/700,168

(22) PCT Filed: Oct. 12, 2022
(86) PCT No.: PCT/US2022/046385

§ 371 Date: Apr. 10, 2024

Related U.S. Application Data
(60) Provisional application No. 63/254,936, filed on Oct. 12, 2021.

(51) Int. Cl. A61M 5/142

(52) U.S. Cl. CPC A61M 5/14244; A61M 2205/10; A61M 2205/3561; A61M 2205/3592; A61M 2205/502; A61M 2205/584; A61M 2205/587; A61M 2205/6081

(57) ABSTRACT

Embodiments of the present disclosure enable a user-wearable infusion pump that may have a limited user interface including no display to be paired with a remote control device that can include a remote consumer electronic device such as a smartphone and/or a dedicated remote controller.

### RELATED APPLICATION

The present application claims the benefit of U.S. Provisional Application No. 63/254,936, filed Oct. 12, 2021, which is hereby incorporated herein in its entirety by reference.

### TECHNICAL FIELD

The present invention relates to medical pumps for delivering medicament to a patient, and more specifically, to a user-wearable pump controllable with a remote control device.

### BACKGROUND

There are many applications in academic, industrial, and medical fields that benefit from devices and methods that are capable of accurately and controllably delivering fluids, such as liquids and gases, that have a beneficial effect when administered in known and controlled quantities. Such devices and methods can be particularly useful in the medical field where treatments for many patients include the administration of a known amount of a substance at predetermined intervals.

One category of devices for delivering such fluids is that of insulin injecting pumps that have been developed for the administration of insulin for those suffering from both Type 1 and Type 2 diabetes. Some insulin injecting pumps configured as portable infusion devices can provide continuous subcutaneous insulin injection and/or infusion therapy for the treatment of diabetes. Therapy may include the regular and/or continuous injection or infusion of insulin into the skin of a person suffering from diabetes and offer an alternative to multiple daily injections of insulin by an insulin syringe or an insulin pen. These pumps can be ambulatory/portable infusion pumps that are worn by the user and may use replaceable cartridges. Medicaments other than or in addition to insulin, such as glucagon, pramlintide, etc. can also be delivered. Examples of such pumps and various features that can be associated with such pumps include those disclosed in U.S. Patent Application Publication No. 2013/0053816, U.S. Pat. Nos. 8,573,027, 8,986,253, U.S. Patent Application Publication No. 2013/0324928, U.S. Patent Application Publication No. 2013/0331790, U.S. Pat. No. 8,287,495 and U.S. patent application Ser. No. 15/158,125, each of which is hereby incorporated herein by reference in its entirety.

One type of pump that has been developed is a patch pump, or micro pump. Patch pumps are small pumps, typically ambulatory, that may be carried directly on the skin under the user's clothing. In some cases, the pumps are situated directly on, or very near to, the injection site such that little or no tubing is required to deliver the insulin or other medicament to the patient. Some patch pumps include a single button on the pump to initiate delivery of medicament and do not include a built-in display or user interface. These pumps are therefore primarily remote-controlled. Having only a single button on the pump provides the advantage of being more robust for waterproofing and resistance to external contaminants. However, a disadvantage is that the functionality of a pump with a single button only is limited without the use of a remote control apparatus, typically including a user interface.

With the proliferation of handheld electronic devices, such as mobile phones (e.g., smartphones), there is a desire to be able to remotely utilize such devices, as well as dedicated wireless controllers designed to work with one or more infusion pumps and/or types of infusion pumps, to optimize usage of infusion pumps. These remote controllers would enable a pump to be monitored, programmed and/or operated more privately, more conveniently and more comfortably. Accordingly, one potential use of dedicated remote devices and handheld consumer electronic devices (such as smartphones, tablets and the like) is to utilize such devices as controllers for remotely programming and/or operating infusion pumps.

To use a smartphone or dedicated remote control device to control an infusion pump, the device must be paired with the particular pump in order to enable the pump to execute commands sent by the remote device. However, with patch pumps described above that may not have a display or robust user interface, there is a limited ability to enter input into the pump to pair the pump with the remote.

### SUMMARY

Embodiments of the present disclosure enable an ambulatory infusion pump that may have a limited user interface including no display to be paired with a remote control device that can include a remote consumer electronic device such as a smartphone and/or a dedicated remote controller. Because the pump does not have a display screen, it can be difficult to securely pair the remote control device with the pump. The system can therefore utilize one or more indicator lights to provide secure pairing by presenting e.g., different colors, patterns, tec. and requiring the user to enter the displayed light sequence into the remote control device.

In an embodiment, a method of pairing a user-wearable infusion pump including one or more indicator lights and no display screen with a remote control device can include receiving input at the remote control device to initiate a pairing procedure for pairing the remote control device with the user-wearable infusion pump. A series of different light patterns can be presented with the indicator lights of the user-wearable infusion pump and instructions can be presented on the remote control device for the user to enter each of the series of different light patterns presented with the indicator lights of the user-wearable infusion pump into the remote control device. If it is verified that each of the different light patterns entered by the user matches each of the different light patterns presented with the indicator lights of the user-wearable infusion pump the remote control device can be paired with the user-wearable infusion pump.

In an embodiment, a system for pairing an infusion pump with a remote control device can include a user-wearable infusion pump including one or more indicator lights and no display screen and a remote control device including a display screen and configured to remotely control the user-wearable infusion pump. The remote control device can be configured to receive input to initiate a pairing procedure for pairing the remote control device with the user-wearable infusion pump and to present instructions on the remote control device for the user to enter each of a series of different light patterns presented with the indicator lights of the user-wearable infusion pump into the remote control device. If it is verified that each of the different light patterns entered by the user matches each of the different light patterns presented with the indicator lights of the user-wearable infusion pump the remote control device can be paired with the user-wearable infusion pump.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1C depicts an embodiment of a pump system according to the disclosure.

FIGS. 2A-2C depict an embodiment of a pump system according to the disclosure.

FIG. 3 depicts an embodiment of a pump system according to the disclosure.

FIGS. 4A-4B depict remote control devices for a pump system according to the disclosure.

FIGS. 5A-5F depict various display screens for an ambulatory infusion pump system according to the disclosure.

FIGS. 6A-6H depict a sequence of menu screens for pairing an ambulatory infusion pump with a remote control device according to the disclosure.

FIGS. 7A-7F depict a sequence of menu screens for pairing an ambulatory infusion pump with a remote control device according to the disclosure.

FIG. 8 depicts a flowchart of method steps for pairing a remote control device with a user-wearable infusion pump having no display screen and one or more indicator lights according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

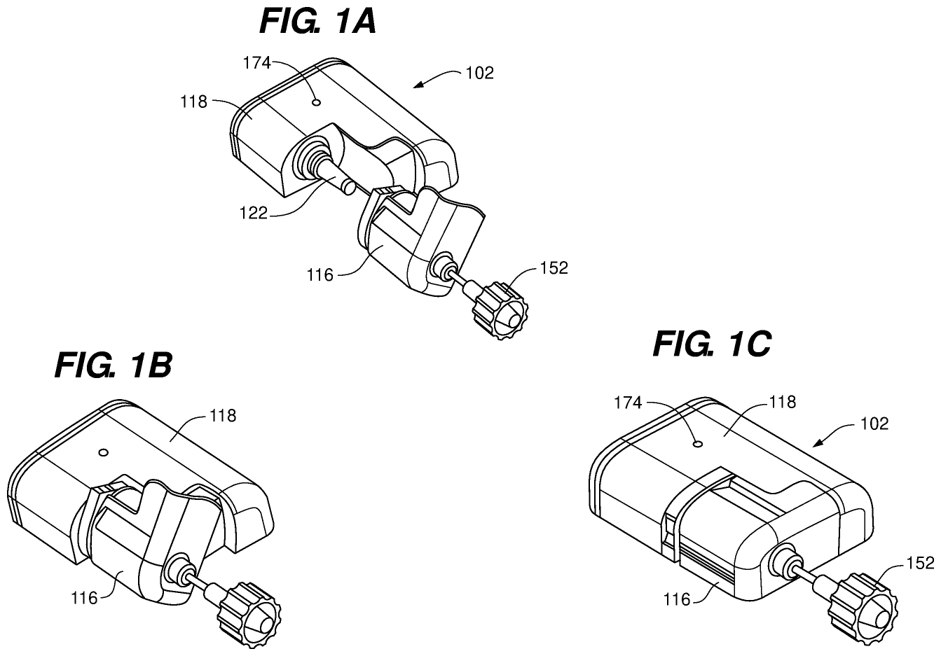

Referring to FIGS. 1A-1C, a pump system 100 including a pump 102 is depicted in accordance with an embodiment of the disclosure. Drive unit 118 of pump 102 includes a drive mechanism 122 that mates with a recess in medicament cartridge 116 of pump 102 to attach the medicament cartridge 116 to the drive unit 118. Further details regarding example embodiments of such delivery mechanisms can be found in U.S. Patent Publication No. 2017/0049957, which is hereby incorporated by reference in its entirety.

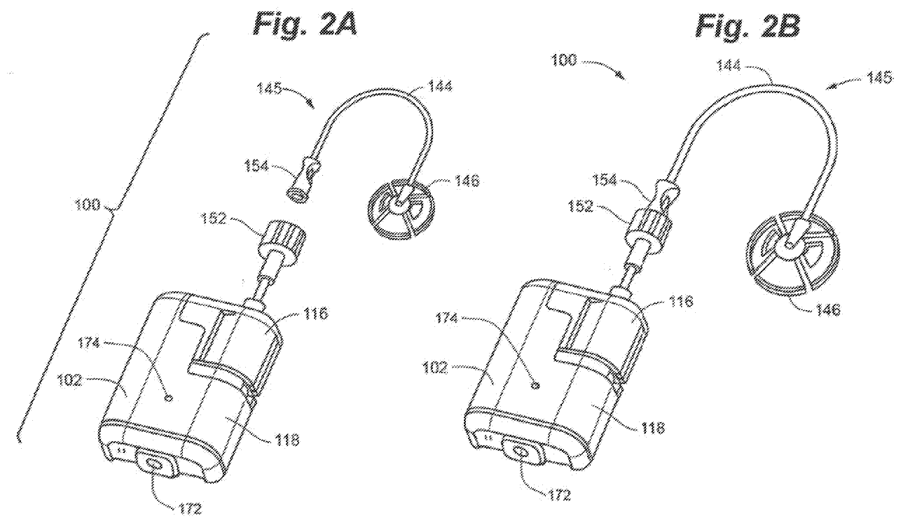

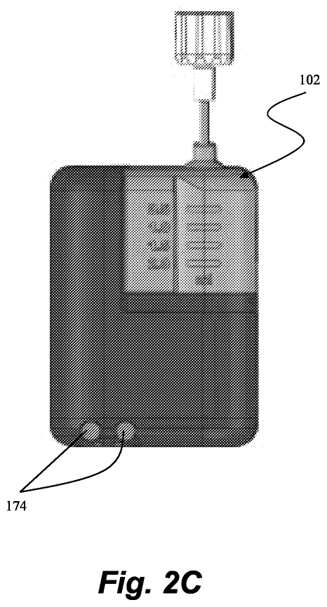

As depicted in the embodiment of FIGS. 2A-2B, pump system 100 can include a pump 102 and an infusion set 145. FIG. 2A depicts this infusion set 145 as not connected to pump while FIG. 2B depicts infusion set 145 connected to pump 102 via connectors 154 and 152. Infusion set 145 can include tubing 144 extending between a connector 154 and a site connector 146. Connector 154 can be configured to couple to pump 102 at connector 152. Site connector 146 can be configured to be attached to an infusion site on a user, while pump 102 can be carried in a separate location, such as the user's pocket or another location on the user's body. Various lengths of tubing 144 can be used in this embodiment to accommodate the user's preference. Further details regarding such pumps can be found in U.S. Pat. Nos. 9,993,595; 10,279,106; and 10,279,107, each of which is hereby incorporated herein by reference in its entirety.

In one embodiment, pump 102 includes a processor that controls operations of the pump and, in some embodiments, may communicate in either one-way or two-way modes to, e.g., receive operational commands and/or other signals, including data, from a separate device and/or, e.g., to send signals, including data, to a separate device. Pump 102 can include one or more buttons configured to cause the processor to initiate one or more functions. In the depicted embodiment, pump 102 includes only a single button 172, although more than one button may be present on pump 102. Button 172 can be configured to, for example, initiate delivery of medicament. Any single button such as button 172 can be utilized to execute a plurality of functions or operations. For example, a single press of button may initiate one function, holding the button down for a predetermined period of time may initiate another function, etc. Because the depicted pump 102 optionally does not itself include a display or user interface, information and feedback regarding medicament delivery or dosing initiated with button 172 can be communicated to and displayed on a remote control device or other device having a display and/or other type of user interface. Further details regarding use of button 172 can be found in U.S. Patent Publication No. 2018/0193555, which is hereby incorporated by reference in its entirety.

In an embodiment, pump 102 includes a light source, such as a light emitting diode (LED) 174. Light source 174 can be configured to provide user feedback regarding user input and/or the performance of a desired function. For example, in one embodiment, light source 174 can illuminate or blink one or more times to indicate that the one or more buttons 172 have been activated and/or that a desired function has been initiated. In one embodiment, pump 102 can additionally and/or alternatively vibrate and/or provide audible notifications to indicate that the one or more buttons 172 have been activated and/or that a desired function has been initiated or, e.g., to provide user feedback regarding user input and/or the performance of the desired function. Illumination of light source 174 and/or vibrations and/or audible notifications may be executed in any number of patterns, frequencies, durations, sequences, combinations, colors, brightness levels, etc. to indicate particular information, such as particular input received and/or particular functions or operations enabled and/or initiated, to the pump user or caregiver. FIG. 2C depicts another embodiment of a pump 102 that includes two indicator lights 174.

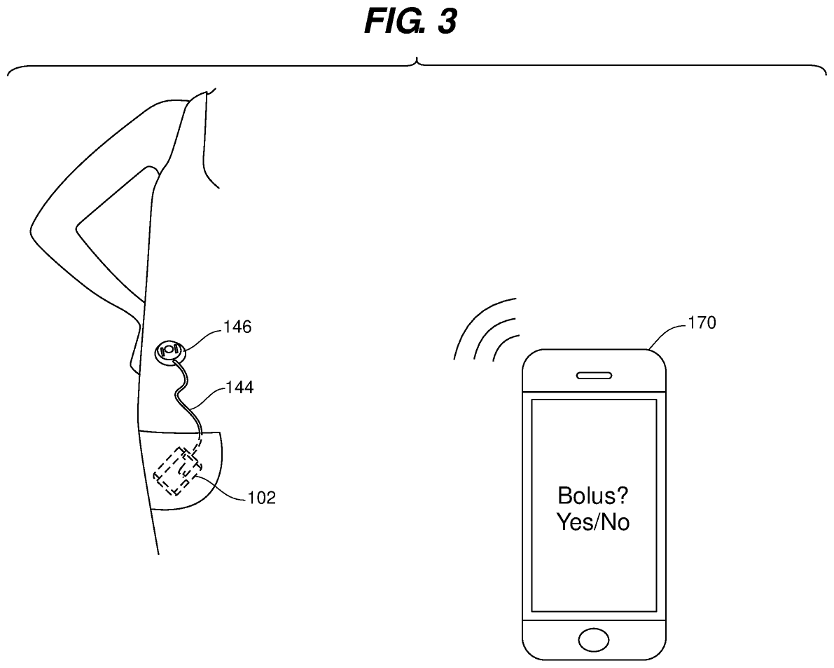

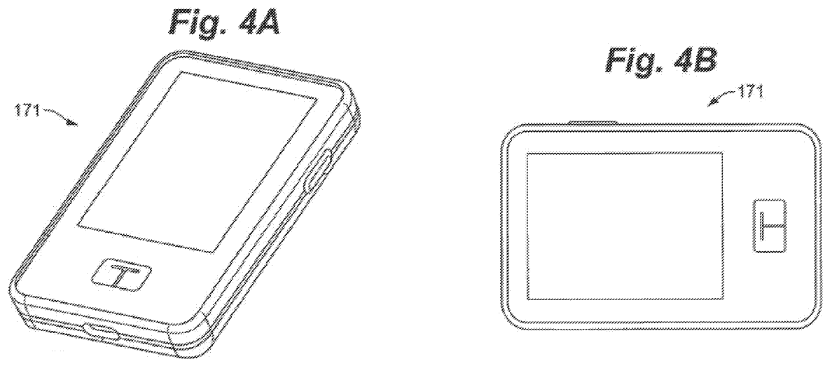

Referring to FIGS. 3-4B, one or more remote control devices 170, 171 can be used to communicate with the processor of pump 102 to control delivery of medicament and transfer data with pump 102 via a wired or a wireless electromagnetic signal, such as via, e.g., a near field communication (NFC) radio frequency (RF) modality or other RF modalities such as Bluetooth®, Bluetooth® low energy, mobile or Wi-Fi communication protocols, for example, according to embodiments of the present disclosure. Such a remote control can include, for example, a mobile communication device 170, such as a smart phone (as depicted in FIG. 3) executing a software application for control of the pump, a dedicated remote controller 171 (as depicted in FIGS. 4A-4B), a wearable electronic watch or electronic health or fitness monitor or personal digital assistant (PDA), etc., or a tablet, laptop or personal computer. Such communications between (and among) the one or more remote control devices 170, 171 and pump 102 may be one-way or two-way for, e.g., effective transfer of data among the devices and the pump, control of pump operations, updating software on the devices and/or pump, and allowing pump-related data to be viewed on the devices and/or pump.

For a pump 102 to be able to be controlled by a remote control device such as a smartphone 170 operating a software application for controlling the pump or dedicated remote controller 171 via wireless communication such as, for example, Bluetooth, the pump must be securely paired with the control device to establish the mutual connection between the devices. For pumps having display screens this can be done by displaying an alphanumeric code on the pump and having the user enter the code into the remote control. However, due to the limited communication capabilities of some user-wearable infusion pumps such as those depicted in FIGS. 1A-1C and 2A-2C that do not include a display screen, it may be difficult to cause the pump to participate in the pairing process and to follow the status of the pump during pairing. Embodiments of the present invention therefore employ additional elements of the system to aid in the pairing process. As noted above, pump 102 may have one or more indicator lights 174 capable of being illuminated in different colors and/or patterns. These lights can therefore be employed in a manner similar to an alphanumeric code to display a specific sequence of light combinations that are entered into the remote control device to mimic the secure pairing provided by such a code displayed on a pump screen.

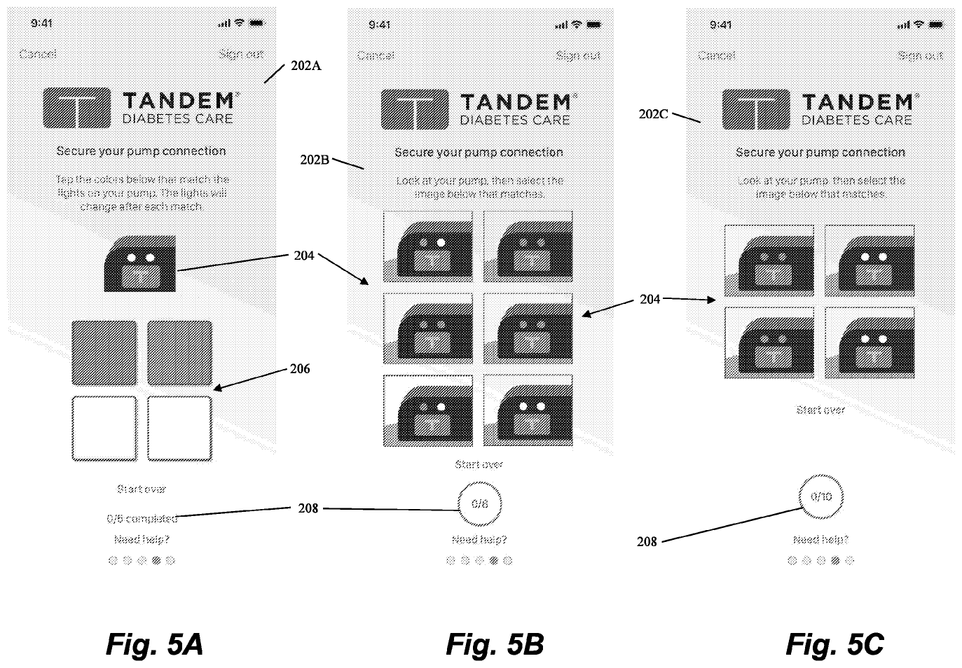

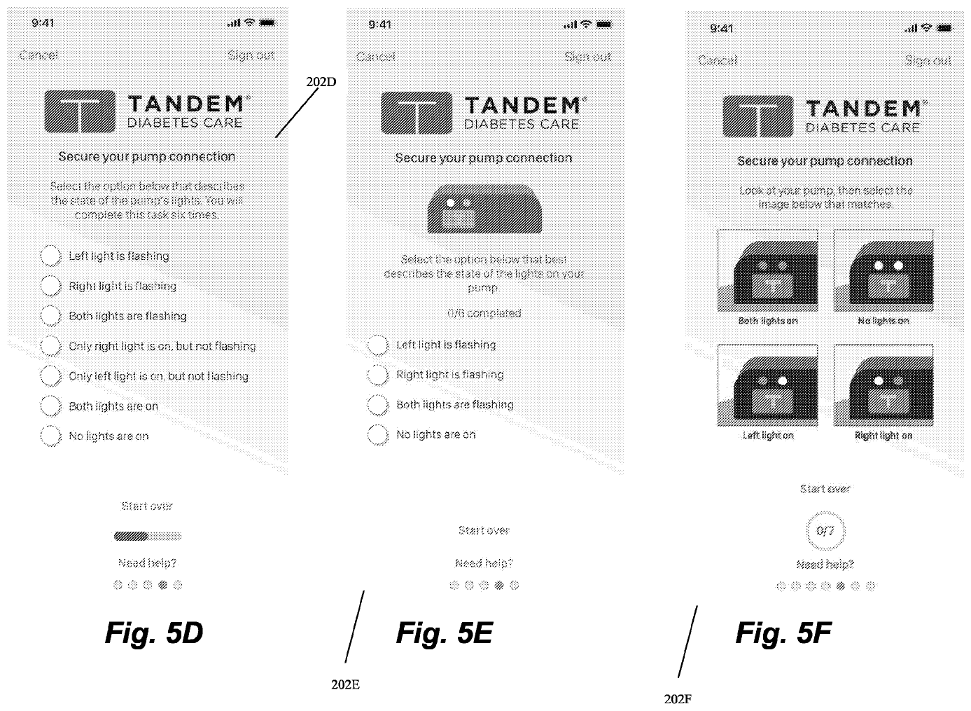

FIGS. 5A-5F depict display screens that can be displayed on the remote control device 170, 171 for pairing the control device with a pump according to various embodiments. Referring to FIG. 5A, pairing screen 202A can include a pump icon 204 and a plurality of selectable color objects 206 (for example, red, blue, yellow and white or off objects). The screen can include instructions for the user to look at the actual indicator lights 174 on the user's pump and to select the color objects 206 corresponding to the colors displayed by the indicator lights. A progress indicator 208 shows that, in this embodiment, the user must enter 5 different light combinations displayed by the pump to complete the pairing process. In the embodiment of FIG. 5B, rather than selectable color objects 206, the pairing screen 202B displays a plurality of selectable pump objects 204 displaying different color combinations (e.g., blue/white, red/red, red/blue, etc.) and the user is instructed to select the pump object 204 that matches the indicator lights 174 on the pump a specific number of times (i.e., 8 times). The pairing screen 202C of FIG. 5C is similar to pairing screen 202B, but includes fewer pump objects 204 (i.e., both lights being the same color in each object) and requires a greater number of matches (i.e., 10 matches) as indicated by the progress indicator 208. FIGS. 5D-5E depict pairing screens 202D, 202E that, rather than having a user enter a color of the indicator lights, have the user enter whether one or more of the lights is flashing, off, on or some combination thereof. In some embodiments, a combination of a color and a type of illumination (i.e., flashing) can be employed. FIG. 5F depicts another pairing screen 202F that does not require color. In this embodiment, either both lights are on, both lights are off, or one of the two lights is on and the user selects the matching image on the screen for each pattern in the sequence.

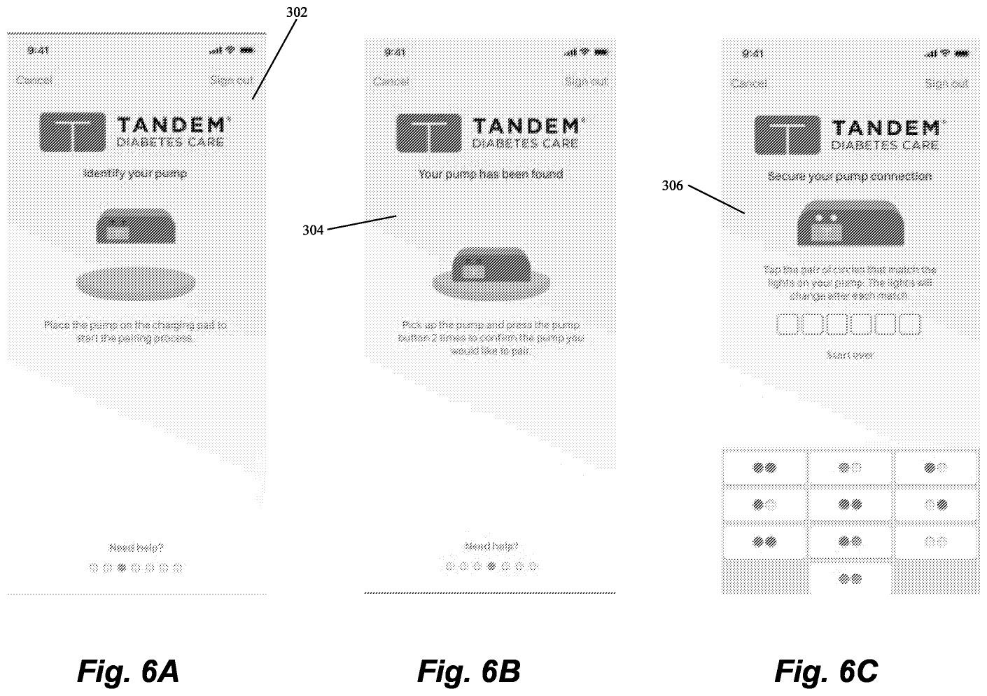

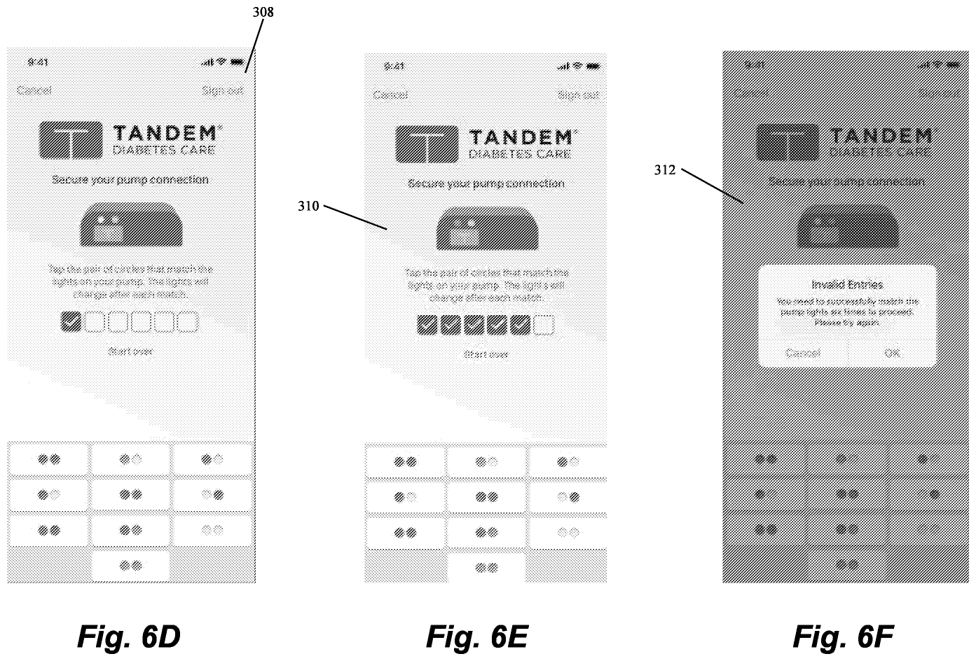

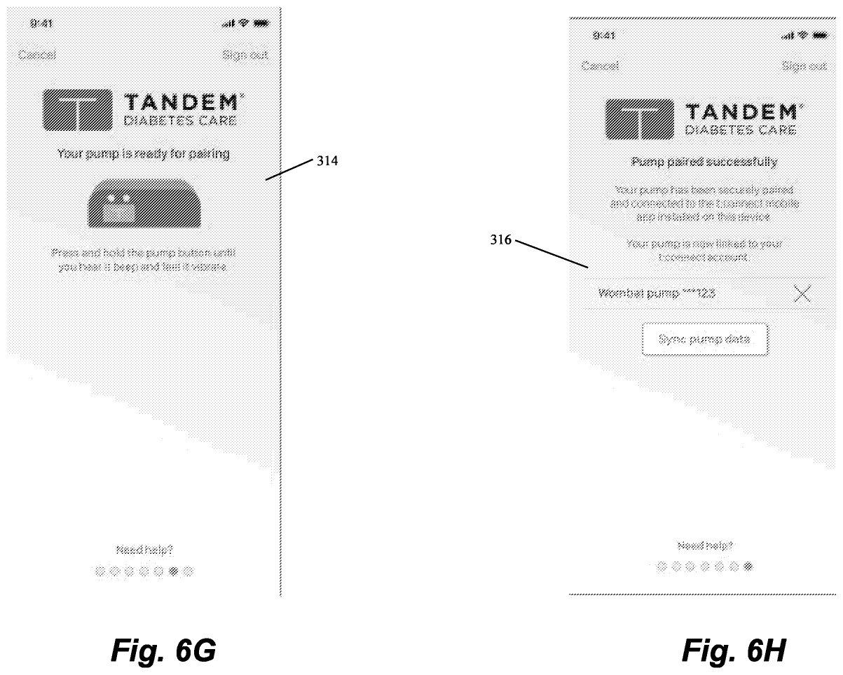

FIGS. 6A-6H schematically depict a sequence of display screens for pairing a remote control device 170, 171 with an infusion pump 102 according to an embodiment. In various embodiments, the pairing sequence can utilize an inductive charging pad 180 (or other charging device) to aid in the pairing process as described in U.S. Pat. No. 10,888,655, which is hereby incorporated by reference in in its entirety. After initiating a pairing procedure through selection of a pairing menu item, the remote control device can initially display a detection screen 302 instructing the user to initiate the pairing procedure by, e.g., placing the pump on the inductive charging pad so the remote can locate the pump followed by an action screen 304 instructing the user to perform an action with the pump, e.g., pressing a pump button 172 two times, to confirm the pump that the user is pairing with the remote control device.

Once the user has confirmed the pump for pairing, the remote control can display an initial pairing screen 306. In this embodiment, pairing screen 306 includes a plurality of different light color combinations with an instruction to the user to tap the pair of lights/circles that match the currently displayed lights on the pump (although any of the pairing screen embodiments described herein could be employed). Pairing screens 308 and 310 depict the user's progress through the pairing sequence, with pairing screen 308 depicting that the user has successfully made one of the six required color matches and display screen 310 depicting that the user has made five of the six required matches. In some embodiments, the color of the lights on the pump depicted on the pairing screens can change each time the user successfully matches the light combination to display the most recent combination. Error screen 312 depicts an error message that can be displayed when the user selects a circle/light combination that does not match the pump. In some embodiments, after acknowledging the error the user can return to the same point in the pairing procedure as when the error occurred. Alternatively, the user may be required to start the matching sequence from the beginning following the error. FIG. 6G depicts a ready screen 314 that can be displayed once the user has successfully completed the matching sequence and informing the user that the devices are ready for pairing and providing an instruction to the user to perform an action to pair the devices, such as, for example, holding a button 172 on the pump until it beeps and/or vibrates. If the pump is successfully paired, a pairing confirmation screen 316 can be displayed.

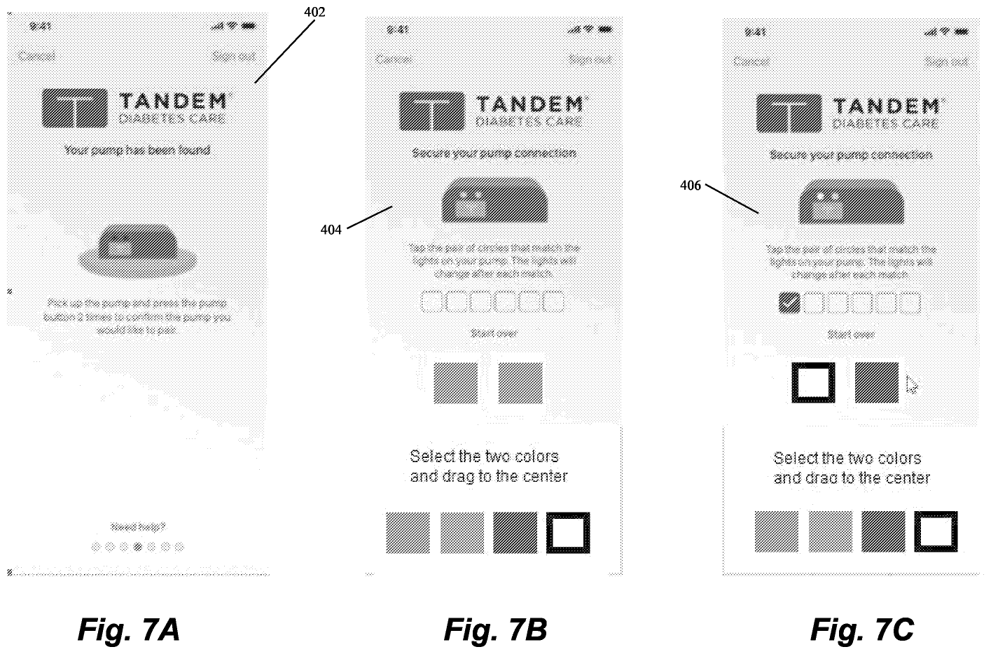

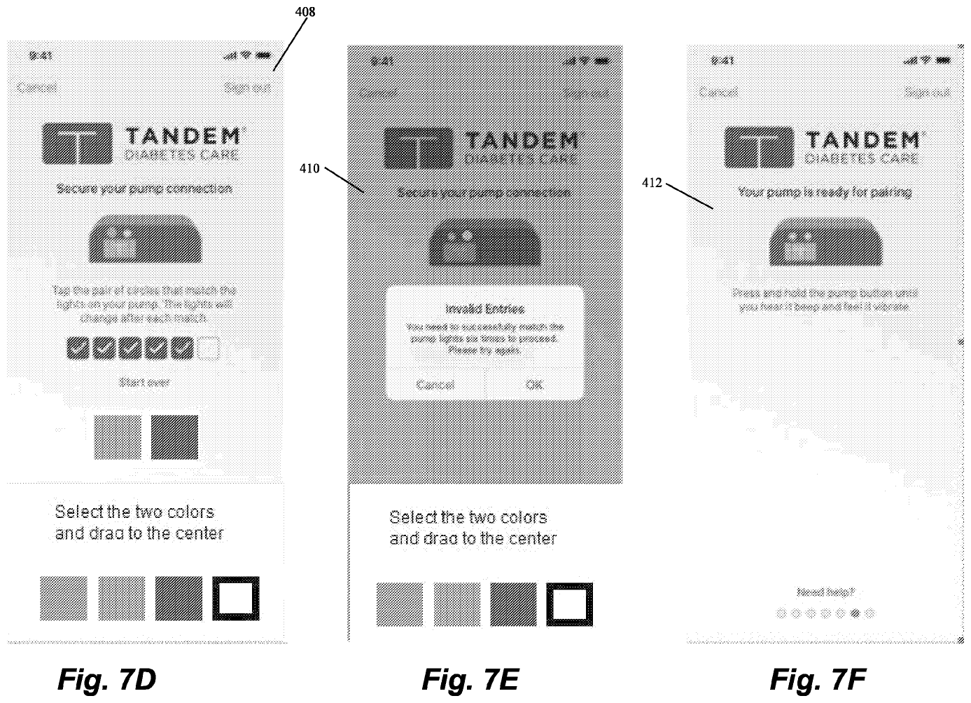

FIGS. 7A-7F schematically depict a sequence of display screens for pairing a remote control device 170, 171 with an infusion pump 102 according to another embodiment. As with the previous embodiment, an action screen 402 can include an instruction for a user to take an action to activate the pairing sequence. In this embodiment, pairing screens 404-408 instruct the user to drag the colors (e.g., green, orange, blue and white or off) displayed at the bottom of the screens to the two available areas in the center of the screen. Alternatively, the user may be able to tap on a color and have it appear on the screen. An incorrect match can also cause the remote control to display an error screen 410. Once the user has completed the matching sequence, a similar ready for pairing screen 412 can be displayed with final pairing instructions and a confirmation screen (not pictured) can be displayed once the devices are paired.

In embodiments, various different colors, color combinations, or other patterns, etc. can be employed. For example, with four different colors (or three colors and fourth color indicated by the light being off) there are 16 different color combinations that can be randomly generated. Alternatively, a set of predefined combinations, e.g., 10 combinations as depicted in FIG. 6C-6E, can be displayed. In addition, in various embodiments the order in which the colors are displayed must be matched (i.e, for two indicator lights) and in other embodiments the user need only match the colors in any order. The number of matches the user must make to complete the pairing process can also vary. For a pump having a display screen that is paired with an, e.g., 6-digit alphanumeric code using only the numeric values 0-9 as described above, the number of total combinations for the code is 10\hat{ }6=1,000,000. In various embodiments, each of the parameters described above for presenting a pairing passcode with the indicator lights of a pump with no display screen can be varied to provide a similar level of security. For example, in the scenario noted above with 4 colors and the order of the colors is required to be matched for 16 different color combinations, requiring the user to enter 5 matches results in 16\hat{ }5=1,048,576 combinations. If the order of the 4 colors does not matter, there are 10 combinations (same as the 0-9 numeric values) and therefore also 1,000,000 total combinations if the user is required to match 6 times. If 3 colors are employed with order not mattering for 6 total combinations, requiring 8 matches yields 6\hat{ }8=1,679,616 matches. If the order of 3 colors must be matched for 9 total combinations, requiring seven matches results in 9\hat{ }7=4,782,969 combinations (6 matches would be 9\hat{ }6=531,441 combinations). In some embodiments, only one color may be shown at a time (and a user would therefore only be required to enter a single color). In such an embodiment having 4 colors requiring 10 matches results in 4\hat{ }10=1,048,756 combinations. Such embodiments could be employed with pumps having only a single indicator light, or could be employed with dual-light configurations by illuminating both lights with the single color.

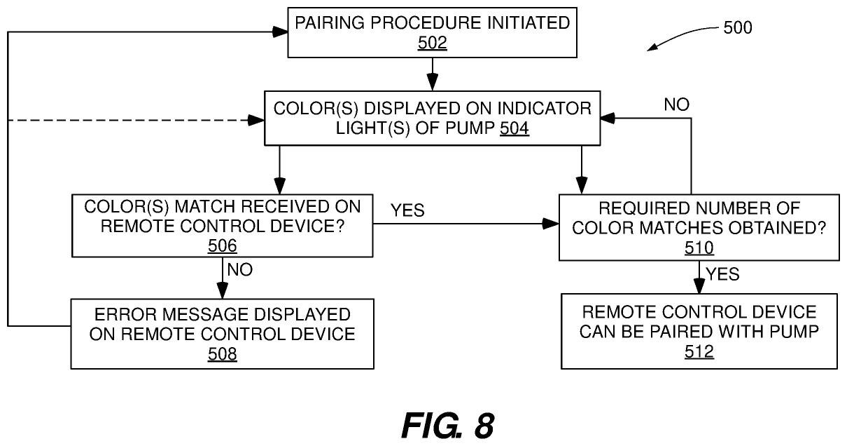

Referring now to FIG. 8, a flowchart of method steps 500 for pairing a remote control device with a user-wearable infusion pump having no display screen and one or more indicator lights is depicted. At step 502, the pairing procedure is initiated. In response, one or more colors are displayed by the indicator light(s) of the pump at step 504 with a corresponding user interface for entering the displayed color(s) displayed on the remote control device consistent with one or more of the embodiments described above. At step 506, it is determined whether the color(s) entered into the user interface match the color(s) displayed by the indicator lights. If the entry is not a match, at step 508 an error message can be displayed on the remote control device. In some embodiments (as indicated by the solid arrow), the system can then revert to the beginning of the pairing procedure (i.e., if the user had made 3 of 6 matches, the user would be required to start over and make 6 more matches following the error). In other embodiments (as indicated by the dashed arrow), the system can revert back to displaying one or more color(s) without starting the procedure over (i.e., if the user had made 3 of 6 matches, the user would only be required to make 3 more matches following the error). If the user entry does match the color(s) of the indicator lights at step 506, it is determined whether the required number of matches to complete the pairing procedure have been obtained. If not, the system reverts back to step 504 and a new color or colors are displayed. If the required number of matches has been obtained, the matching sequence has been completed and at step 512 the remote control device can proceed to being paired with the pump. Although FIG. 8 describes a pairing procedure that employs colors of LED lights on a pump, as noted above in other embodiments the lights can indicate a sequence of patterns that does not rely on color.

In some embodiments, the indicator lights on the pump can be employed to correspond with a number for a pairing code. For example, a sequence may occur where a pump first blinks three times requiring the user to input the number “3” into the pairing screen, followed by, e.g., the light blinking two times, then one time, then 5 times for a pairing code of 3-2-1-5 (although a code of any number of digits could be used). Such a system could also be paired with color with each blinking pattern corresponding to a color and the user being required to select a corresponding color along with entering the number.

In other embodiments, the pump can utilize noise alternatively or in addition to the indicator lights to provide a pairing passcode. For example, similar to the embodiment above in which the lights flash a certain number of times for each number in a code, the pump can beep or otherwise provide a sound a discrete number of times to indicate each number in the code. Alternatively, the device could provide specific sounds that must be matched to corresponding icons displayed on the pairing screen. In one embodiment, the pump could provide animal sounds and the user would match each sound with a corresponding animal, such as, e.g., matching a moo sound with a cow, a quack sound with a duck, a bark sound with a dog and an oink sound with a pig, etc.

Systems and methods described herein therefore can provide a secure pairing process that approximates the use of a passcode presented on a display screen of an infusion pump by using the one or more indicator lights of a pump to transmit the code without using a communications network (i.e. “out of band”). The pairing process is user-friendly requiring minimal user interaction and employing an auto-progressing workflow that can be completed in minimal time while maintaining a required level of security. The disclosed method of pairing also provides advantages over a predetermined passcode in that there is no risk of a user losing the device passcode. In some embodiments, the disclosed pairing procedures can be a selective mode that can be turned on and off by the user. For example, in one embodiment a passcode may be the primary or default pairing procedure and a user can access a menu item that enables pairing by one or more of the disclosed procedures.

In embodiments, colors can be chosen to aid colorblind patients and/or caregivers in completing the pairing procedure. For example, the most common form of color blindness is red-green color blindness that makes it difficult to differentiate between red and green. As such, only red or green would be employed in the color matching scheme and not both colors. The combination of colors can selectively be chosen to provide a set of colors that is most easily differentiated by colorblind individuals. In some embodiments, an option may be presented to the user to change the array of colors being displayed for the pairing procedure to a new array of colors that the user may select if the user is colorblind or otherwise is having difficulty distinguishing between the displayed colors.

In another embodiment, rather than have a user manually enter the displayed combinations via the user interface of the remote control device, for control devices such as smartphones having cameras the user can use the camera to scan the indicator lights of the pumps to “read” the pattern of the lights. In addition to eliminating the potential for data entry errors, such an embodiment would further provide an advantage of aiding colorblind patients in embodiments that vary the color of the lights.

As noted above, illumination of the one or more indicator lights 174, and particularly in a pump such as that in FIG. 2C having two indicator lights 174, can be executed in any number of patterns, frequencies, durations, sequences, combinations, colors, brightness levels, etc. to indicate particular information. The examples given above with respect to various colors, illumination patterns and combinations, etc. are illustrative only and it should be understood that different information can be conveyed by varying the output of indicator lights 174 in any number of ways.

In embodiments, a method of pairing a user-wearable infusion pump including one or more indicator lights and no display screen with a remote control device can include receiving input at the remote control device to initiate a pairing procedure for pairing the remote control device with the user-wearable infusion pump. A series of different light patterns can be presented with the indicator lights of the user-wearable infusion pump and instructions can be presented on the remote control device for the user to enter each of the series of different light patterns presented with the indicator lights of the user-wearable infusion pump into the remote control device. If it is verified that each of the different light patterns entered by the user matches each of the different light patterns presented with the indicator lights of the user-wearable infusion pump the remote control device can be paired with the user-wearable infusion pump.

In some embodiments, presenting a series of different light patterns includes displaying different color combinations with the indicator lights.

In some embodiments, presenting instructions on the remote control device for the user to enter each of the series of different light patterns presented with the indicator lights of the user-wearable infusion pump into the remote control device includes displaying a plurality of different individual color objects and instructing the user to select the color objects matching a currently displayed light pattern.

In some embodiments, presenting instructions on the remote control device for the user to enter each of the series of different light patterns presented with the indicator lights of the user-wearable infusion pump into the remote control device includes displaying a plurality of different light combinations and instructing the user to select a light combination matching a currently displayed light pattern.

In some embodiments, presenting a series of different light patterns includes varying whether one or more indicator lights are on, off or flashing.

In some embodiments, presenting instructions on the remote control device for the user to enter each of the series of different light patterns presented with the indicator lights of the user-wearable infusion pump into the remote control device includes displaying text relating to combinations of the one or more indicator lights being on, off or flashing and instructing the user to select the text corresponding to a currently displayed light pattern.

In some embodiments, presenting instructions on the remote control device for the user to enter each of the series of different light patterns presented with the indicator lights of the user-wearable infusion pump into the remote control device includes displaying icons depicting combinations of the one or more indicator lights being on or off and instructing the user to select the icon corresponding to a currently displayed light pattern.

In some embodiments, presenting instructions on the remote control device for the user to enter each of the series of different light patterns presented with the indicator lights of the user-wearable infusion pump into the remote control device includes updating a status of the pairing procedure on the remote control device.

In some embodiments, updating the status of the pairing procedure on the remote control device includes updating the status each time a light pattern entered by the user matches a light pattern presented with the indicator lights of the user-wearable infusion pump.

In some embodiments, presenting instructions on the remote control device for the user to enter each of the series of different light patterns presented with the indicator lights of the user-wearable infusion pump into the remote control device includes presenting an error message if a light pattern entered by the user does not match a light patterns presented with the indicator lights of the user-wearable infusion pump.

In an embodiment, a system for pairing an infusion pump with a remote control device can include a user-wearable infusion pump including one or more indicator lights and no display screen and a remote control device including a display screen and configured to remotely control the user-wearable infusion pump. The remote control device can be configured to receive input to initiate a pairing procedure for pairing the remote control device with the user-wearable infusion pump and to present instructions on the remote control device for the user to enter each of a series of different light patterns presented with the indicator lights of the user-wearable infusion pump into the remote control device. If it is verified that each of the different light patterns entered by the user matches each of the different light patterns presented with the indicator lights of the user-wearable infusion pump the remote control device can be paired with the user-wearable infusion pump.

In some embodiments, the user-wearable infusion pump is configured to present a series of different light patterns by displaying different color combinations with the indicator lights.

In some embodiments, the processor is configured to present instructions on the remote control device for the user to enter each of the series of different light patterns presented with the indicator lights of the user-wearable infusion pump into the remote control device by displaying a plurality of different individual color objects and instructing the user to select the color objects matching a currently displayed light pattern.

In some embodiments, the processor is configured to present instructions on the remote control device for the user to enter each of the series of different light patterns presented with the indicator lights of the user-wearable infusion pump into the remote control device by displaying a plurality of different light combinations and instructing the user to select a light combination matching a currently displayed light pattern.

In some embodiments, the user-wearable infusion pump is configured to present a series of different light patterns by varying whether one or more indicator lights are on, off or flashing.

In some embodiments, the processor is configured to present instructions on the remote control device for the user to enter each of the series of different light patterns presented with the indicator lights of the user-wearable infusion pump into the remote control device by displaying text relating to combinations of the one or more indicator lights being on, off or flashing and instructing the user to select the text corresponding to a currently displayed light pattern.

In some embodiments, the processor is configured to present instructions on the remote control device for the user to enter each of the series of different light patterns presented with the indicator lights of the user-wearable infusion pump into the remote control device by displaying icons depicting combinations of the one or more indicator lights being on or off and instructing the user to select the icon corresponding to a currently displayed light pattern.

In some embodiments, the processor is further configured to update a status of the pairing procedure on the display screen of the remote control device.

In some embodiments, the processor is configured to update the status of the pairing procedure by updating the status each time a light pattern entered by the user matches a light pattern presented with the indicator lights of the user-wearable infusion pump.

In some embodiments, the processor is further configured to present an error message if a light pattern entered by the user does not match a light pattern presented with the indicator lights of the user-wearable infusion pump.

Although the pump system described herein is described as a user-wearable pump system that has no display or user interface and is primarily controlled by a remote device, it should be understood that aspects of the present disclosure can be incorporated into other types of infusion pumps. For example, full-featured user-wearable infusion pumps having display and input capabilities, such as a touchscreen display on the pump housing, one example of which is disclosed in U.S. Pat. No. 8,287,495, which is hereby incorporated by reference herein, can incorporate aspects of the present disclosure.

Although the embodiments herein have been specifically described with respect to an ambulatory infusion pump, the inventions disclosed herein could be employed with any other type of programmable medical device capable of receiving and executing remote commands. Such devices include, for example, non-ambulatory pumps, implantable pumps, defibrillators, spinal cord stimulation systems, etc. Embodiments could further include non-medical applications.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; and 11,464,908 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0154405; 2021/0353857; 2022/0062553; 2022/0139522; 2022/0223250; 2022/0233772; 2022/0233773; 2022/0238201; and 2022/0265927 and commonly owned U.S. patent application Ser. Nos. 17/368,968; 17/729,464; 17/732,208; 17/878,681; 17/879,959; 17/886,998 and 17/896,492.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A method of pairing a user-wearable infusion pump with a remote control device, the user-wearable infusion pump including one or more indicator lights, comprising:
receiving input at the remote control device to initiate a pairing procedure for pairing the remote control device with the user-wearable infusion pump;
presenting a series of different light patterns with the one or more indicator lights of the user-wearable infusion pump;
presenting instructions on the remote control device for the user to enter each of the series of different light patterns presented with the one or more indicator lights of the user-wearable infusion pump into the remote control device;
verifying that each of the different light patterns entered by the user matches each of the different light patterns presented with the one or more indicator lights of the user-wearable infusion pump; and
pairing the remote control device with the user-wearable infusion pump if each of the different light patterns entered by the user matches each of the different light patterns presented with the one or more indicator lights of the user-wearable infusion pump.

2. The method of claim 1, wherein presenting a series of different light patterns includes displaying different color combinations with the one or more indicator lights.

3. The method of claim 2, wherein presenting instructions on the remote control device for the user to enter each of the series of different light patterns presented with the one or more indicator lights of the user-wearable infusion pump into the remote control device includes displaying a plurality of different individual color objects and instructing the user to select the color objects matching a currently displayed light pattern.

4. The method of claim 2, wherein presenting instructions on the remote control device for the user to enter each of the series of different light patterns presented with the one or more indicator lights of the user-wearable infusion pump into the remote control device includes displaying a plurality of different light combinations and instructing the user to select a light combination matching a currently displayed light pattern.

5. The method of claim 1, wherein presenting a series of different light patterns includes varying whether one or more indicator lights are on, off or flashing.

6. The method of claim 5, wherein presenting instructions on the remote control device for the user to enter each of the series of different light patterns presented with the one or more indicator lights of the user-wearable infusion pump into the remote control device includes displaying text relating to combinations of the one or more indicator lights being on, off or flashing and instructing the user to select the text corresponding to a currently displayed light pattern.

7. The method of claim 5, wherein presenting instructions on the remote control device for the user to enter each of the series of different light patterns presented with the one or more indicator lights of the user-wearable infusion pump into the remote control device includes displaying icons depicting combinations of the one or more indicator lights being on or off and instructing the user to select the icon corresponding to a currently displayed light pattern.

8. The method of claim 1, wherein presenting instructions on the remote control device for the user to enter each of the series of different light patterns presented with the one or more indicator lights of the user-wearable infusion pump into the remote control device includes updating a status of the pairing procedure on the remote control device.

9. The method of claim 8, wherein updating the status of the pairing procedure on the remote control device includes updating the status each time a light pattern entered by the user matches a light pattern presented with the one or more indicator lights of the user-wearable infusion pump.

10. The method of claim 1, wherein presenting instructions on the remote control device for the user to enter each of the series of different light patterns presented with the one or more indicator lights of the user-wearable infusion pump into the remote control device includes presenting an error message if a light pattern entered by the user does not match a light pattern presented with the one or more indicator lights of the user-wearable infusion pump.

11. A system for pairing an infusion pump with a remote control device, comprising:
a user-wearable infusion pump including one or more indicator lights and no display screen; and
a remote control device including a display screen and configured to remotely control the user-wearable infusion pump, wherein the remote control device includes a processor configured to:
receive input to initiate a pairing procedure for pairing the remote control device with the user-wearable infusion pump;
present instructions on the remote control device for the user to enter each of a series of different light patterns presented with the one or more indicator lights of the user-wearable infusion pump into the remote control device;
verify that each of the different light patterns entered by the user matches each of the different light patterns presented with the one or more indicator lights of the user-wearable infusion pump; and
pair the remote control device with the user-wearable infusion pump if each of the different light patterns entered by the user matches each of the different light patterns presented with the one or more indicator lights of the user-wearable infusion pump.

12. The system of claim 11, wherein the user-wearable infusion pump is configured to present a series of different light patterns by displaying different color combinations with the one or more indicator lights.

13. The system of claim 12, wherein the processor is configured to present instructions on the remote control device for the user to enter each of the series of different light patterns presented with the one or more indicator lights of the user-wearable infusion pump into the remote control device by displaying a plurality of different individual color objects and instructing the user to select the color objects matching a currently displayed light pattern.

14. The system of claim 12, wherein the processor is configured to present instructions on the remote control device for the user to enter each of the series of different light patterns presented with the one or more indicator lights of the user-wearable infusion pump into the remote control device by displaying a plurality of different light combinations and instructing the user to select a light combination matching a currently displayed light pattern.

15. The system of claim 11, wherein the user-wearable infusion pump is configured to present a series of different light patterns by varying whether one or more indicator lights are on, off or flashing.

16. The system of claim 15, wherein the processor is configured to present instructions on the remote control device for the user to enter each of the series of different light patterns presented with the one or more indicator lights of the user-wearable infusion pump into the remote control device by displaying text relating to combinations of the one or more indicator lights being on, off or flashing and instructing the user to select the text corresponding to a currently displayed light pattern.

17. The system of claim 15, wherein the processor is configured to present instructions on the remote control device for the user to enter each of the series of different light patterns presented with the one or more indicator lights of the user-wearable infusion pump into the remote control device by displaying icons depicting combinations of the one or more indicator lights being on or off and instructing the user to select the icon corresponding to a currently displayed light pattern.

18. The system of claim 11, wherein the processor is further configured to update a status of the pairing procedure on the display screen of the remote control device.

19. The system of claim 18, wherein the processor is configured to update the status of the pairing procedure by updating the status each time a light pattern entered by the user matches a light pattern presented with the one or more indicator lights of the user-wearable infusion pump.

20. The system of claim 11, wherein the processor is further configured to present an error message if a light pattern entered by the user does not match a light pattern presented with the one or more indicator lights of the user-wearable infusion pump.
